(19) United States
(12) Patent Application Publication
(10) Pub. No.: US 2008/0039803 A1
(43) Pub. Date: Feb. 14, 2008

(54) Luer protection pouch™ and luer valve/male luer protection method

(76) Inventor: Lawrence Allan Lynn, Columbus, OH (US)

Correspondence Address: The Sleep and Breathing Research Institute, Suite 10, 1275 Olentangy River Road, Columbus, OH, 43212, US

(21) Appl. No.: 11/805,381

(22) Filed: May 23, 2007

Related U.S. Application Data
(60) Provisional application No. 60/836,637, filed on Aug. 9, 2006.
(60) Provisional application No. 60/900,536, filed on Feb. 8, 2007.

(51) Int. Cl. A61M 39/16

(52) U.S. Cl. 604/256

(57) ABSTRACT

A universal medical Luer Protecting Pouch™ is disclosed. The inexpensive elastic pouch contains disinfectant and is configured to terminate the present epidemic in hospitals by providing a very inexpensive and easily implemented universal device for protecting and swabbing a wide variety of luer systems from bacterial contamination. The pouch has a flattened configuration and can be elastically dilated, as by squeezing the pouch between the thumb and index finger, to allow for simplified cover of both luer valves and male luers with a single device, which is readily carried in large numbers in nurse's pockets (in a manner similar to that for conventional alcohol swabs). The same pouch covers either the face of the luer valve or distal end of the male luer at the discretion of the user and without transmission of torsion or longitudinal force which might loosen the luer valve or otherwise be transmitted to the vein.

This application claims priority of Provisional Application 60/836,637, filed Aug. 9, 2006, and Provisional Application 60/900,536, filed Feb. 8, 2007, the contents of each of which are incorporated by reference as if completely disclosed herein. The contents of application Ser. No. 11/724,812 and application Ser. No. 11/724,888 and the utility application filed on May 6^{th} 2007 and entitled “Luer Valve Disinfectant Swab-Pouch” (all of the present inventor) are incorporated by reference as if completely disclosed herein.

### BACKGROUND AND SUMMARY OF THE INVENTION

For any patient managed with an open piston valve, there is a clear and present danger that the piston face of the valve will be contaminated by the drug resistant bacteria, which are ubiquitous in many hospital wards. There is also a clear and present danger that these bacteria will be injected directly into the patient. In fact, anytime a male luer is pressed against a contaminated piston face of the valve, the male luer actually captures bacteria for direct injection into the bloodstream of a patient. This might be best called a “Direct Bacterial Injection” (DBI).

The open piston luer valves in wide use today (see FIG. 1) commonly have exposed piston faces which are engaged by the large open but initially sterile fluid channel (lumen) at the tip of the male luer during the first access. It is this large open circular end, which captures the bacteria from the face of the piston valve for direct injection into the bloodstream. Furthermore, the open piston valves also have exposed circumferential side walls which have been identified by the CDC as an important source of bacterial incubation. (See arrow FIG. 1)

Not surprisingly, recent evidence suggests that there is an epidemic of hospital acquired bloodstream infections potentially caused by the new generation of mechanical open piston luer valves which are now in the market. It is one of the purposes of the present invention to provide a very simple and very inexpensive device and method to protect both luer valves and the male luers from bacterial contamination and to thereby, promptly end this deadly epidemic.

The present inventor has identified the 15 top potential sources of this epidemic as,
1. Direct injection of bacteria into a patient's bloodstream called by the present inventor an iatrogenic, “Direct Bacterial Injection” (DBI) event due to failure to swab the piston face prior to access, allowing potentially large numbers of bacteria to be trapped within the lumen of the male luer producing this bolus bacterial injection.
2. Failure to swab the piston face causing transfer of large numbers of bacteria may be into circumferential space around the piston as well as the outer wall of the male luer where they can incubate and later contribute to Direct Bacterial Injection events.
3. Ineffective swabbing of the piston face, such as the typical “Single Swipe” allowing the bacteria which remain on the face to be trapped within the lumen of the accessing male luer producing “Direct Bacterial Injection” wherein the bacteria remaining after the swipe can be injected as a bacterial bolus (for example into an immune incompetent patient's bloodstream).
4. Contamination of the outer wall and lumen of the male luer of piggy back systems caused by failure to cap the male luer between piggy back accesses.
5. Colonization of the male luer cap induced by the capping of a contaminated male luer or exposing the interior or the male luer cap to environmental or droplet contamination.
6. Transfer of bacteria from a contaminated male luer and/or male luer cap to the circumferential space around the piston of the valve.
7. Colonization of the space (see Arrow FIG. 1) around the piston and subsequent contamination of the male luer of a repeat-use piggy back system.
8. Contamination of the outer wall of the male luer by contact with contaminated outer edge of the piston housing on removal of the male luer from within housing.
9. The pooling of liquid within the space around the piston allowing increased presence of gram negative bacteria, (this type of bacteria are water lovers and once present can readily incubate in the fluid within the space around the piston or the fluid which is often retained in a male luer cap).
10. Increased presence of bacteria which can be more resistant to alcohol disinfectant (such as certain water loving gram negative rods)
11. The aspiration of nutrient fluids (such as, albumin, parenteral nutrition, blood products and lipids, as for example, with the sedative Propofol, to name a few) into the circumferential space around the piston, with subsequent rapid incubation of large numbers of bacteria and subsequent carriage of these nutrients on the outer wall of the male luer of repeat-use piggy back systems into the luer cap for contemporaneous incubation at that location.
12. Failure to change a piston valve after 72 hours, which allows potential for massive bacterial incubation and biofilm development within the circumferential space around the piston as well as transfer of that biofilm on the outer wall of the male luer to the luer cap.
13. Inadvertent contact of the male luer of a repeat-use piggy back system with contaminated surfaces prior to access and associated carriage of the bacteria into the circumferential space around the piston and into the male luer cap.
14. Auto-contamination of the piston face from the space around the piston during multiple sequential accesses such as sequential saline-drug-saline-heparin flushes, saline flush procedures, or during titration of awake sedation with multiple accesses following a single initial swabbing. (this procedure is called, by the present inventor, “Swab Once, Access Many Times”, (SOAMT) and this procedure is hazardous with open piston valves because the face of open piston valves they can auto-contaminate and then cause a “Direct Bacterial Injection”.
15. Increased potential clinical bacteremia in response to the injection of bacteria, due to increased virulence and antibiotic resistance of the bacteria which are injected by the above means.

In view of this severe and largely uncontrolled risk, it is most extraordinary that hospitals depend on the unpredictable behavior of active cleansing of the piston face to protect the patients from the direct injection of potentially deadly bacteria. It is even more extraordinary that hospitals depend on this unpredictable behavior even in the management of patients with AIDS, prosthetic heart valves, bone marrow transplants, or even with profoundly low white blood cell counts. In many cases the workers know they should swab, they simply do not do it. Like any work environment, personal diligence, even with sufficient understanding and reminders, is not universal. Therefore it is unlikely that this risk can be reasonably controlled by improved methods to remind healthcare workers, as by posters in each room, or by asking the patient to remind the worker to swab. It is clear that the risk of these open piston mechanical valves will not be effectively mitigated by efforts to remind the workers or to otherwise enhance healthcare worker diligence.

Unfortunately, failure to disinfect an open piston valve prior to access poses a much greater risk of direct bacterial bolus injection than was associated with the older small needle and septum systems they replaced. Since the distal exposed circular area of the lumen of a male luer is vastly greater than that of a 19 to 22 gauge beveled needle, capture of bacteria within the lumen of the male luer from a contaminated piston face is vastly greater than that within the lumen of the needle from the face of a penetrable septum. This difference was not fully appreciated when the open piston valves were introduced. Indeed, when the open piston valves were first introduced, physicians and nurses, perceiving the open piston valves as safe for patients as needled systems, began to freely allow the face of piston valves to be completely exposed between accesses to sources of bacterial contamination. No cap was generally applied to protect the face so the face was allowed to contaminate freely. At that time, nurses and physicians did not understand that, bacteria contaminating the faces of these valves, if not removed, are much more readily captured under the large diameter luer tip. Nor did they realize that this capture would place these bacteria within the lumen at the tip of the male luer, when the piston face is engaged by the advancing male luer. Worse, they did not know that, after such capture, these bacteria are directly injected into the patients.

A recent study confirmed the danger of direct large bolus bacterial injection associated with the open piston valves. This study evaluated the potential transmission of bacteria (such as those trapped under the luer tip) into patients when the piston face is not cleansed. In this study, control open piston valves were contaminated and then accessed by male luers without prior cleansing of the piston face. The study found that “all 15 control open piston valves (100%) showed massive transmission of microorganisms across the piston (4,500-10,000 colony-forming units).” See, Maki, et. al. Infect Control Hosp Epidemiol. 2006 January; 27(1):23-7. This potentially fatal event comprises a “bolus bacterial injection” which can occur when the faces of luer piston valves are left unprotected between accesses and the nurse then simply forgets, or is too hurried to swab the face before male luer insertion. As noted previously, in retrospect, this is not surprising since, the open distal lumen of the male luer is quite large and therefore a large number of bacteria located on the piston face are readily trapped within the lumen of the male luer when that lumen is pressed against the piston face.

Another problem is incomplete swabbing. Commonly a single wipe of an alcohol swab across the face of a valve is applied however this may not remove enough bacteria to make the access into the valve safe. (“See again, Maki, et. al. Infect Control Hosp Epidemiol. 2006 January; 27(1):23-7.) Therefore, especially with highly contaminated valve faces or with bacteria which are not very susceptible to alcohol, the act of swabbing actually provides a false sense of security. Clearly the risk of bacterial loading of the valve face between luer accesses cannot be obviated by greater education and diligence, even if more universal swabbing compliance could be achieved.

To understand how hospitals found themselves widely using access systems capable of providing a mechanism for routine direct bolus injection of bacteria directly into patient's blood vessels, it is important to first comprehend the magnitude of the healthcare worker safety initiative of the 1990s. This mandate favored the expansion of the market for luer valves. Initially, open (but capped) luer valves entered the market. These were promoted as “eliminating the needle” but the ports of valves were deeply recessed and could not be internally swabbed. For this reason they were marketed with luer valve caps to cover the port between luer entries into the ports. With these valves, luer access required first, an uncapping procedure and then, a recapping procedure. Uncapping and recapping had not been required with the prior needled systems and this uncapping and recapping (and the need to mind the cap during connection) was not popular with nurses. The luer uncapping and recapping procedure might seem a minimal effort to provide protection from contamination however, because it is to be performed while maintaining the sterility of the open interior of the cap it is somewhat cumbersome. After a sterile luer cap has been removed it is easy to contaminate its interior and easy to drop or contaminate the cap. Nurses often found it difficult to proceed with the luer access procedure while holding the little luer valve cap between the index finger and the thumb to keep its interior from becoming contaminated (as would easily happen if the luer cap is held in a closed hand). There was no widely accepted solution for what to do with the cap during prolonged connections. Furthermore, the routine replacement of an old luer cap with a new sterile luer cap, after each piggy back infusion, resulted in a significant increase in expense and this was not always easy to implement. To make matters worse, nurses were already supposed to cap the male luer after use and this was traditionally a different type of sterile cap.

In response to the unpopularity of uncapping, recapping, and storing or replacing the cap, the open piston valves were introduced. In essence the open piston valves were promoted as allowing a return to the simpler disinfectant swab maneuver which was widely used with the older penetrable split septum and needle systems prior to the introduction of the luer valves. Certainly, at the time, nascent open piston valves seemed like an advantage for both hospitals and nurses since the elimination of the thread able cap reduced global cost and the elimination of the perceived need to uncap, recap, and store or replace the cap reduced the work required for luer access.

Unfortunately throughout the hospital environment there are many sources of contamination of the piston face of luer valves and male luers if the face of the valve and tip of the male luer are not covered between connections. A piston face left exposed will become contaminated. For example, routine contact of the exposed piston face with the skin of the patient or healthcare worker, droplet nuclei from talking, coughing or sneezing, (generated by the patient, visitors, other patients, or the healthcare workers), excretions from nasal drainage, wounds, or nasal gastric tubing (carried inadvertently by hands of the patient or others or by direct contact) all comprises sources of piston face contamination with deadly, drug resistant bacteria. As noted, the pressing of a male luer against a contaminated piston face without prior cleansing will simply capture these deadly organisms within the lumen of the male and, as the above study demonstrated, like discharging a loaded gun to the patient, these bacteria will be injected in a bolus directly into the patient's vascular system as soon as fluid is injected through the male luer. In some patients the simple act of injecting these bacteria will result in irreversible contamination of a prosthesis, debilitating sepsis, and/or death.

Microorganisms are becoming more resistant to antibiotics and factors which greatly amplify the risk to the patient posed by the injection of resistant bacteria into patients due to failure to swab a piston face are increasing. As noted, factors which make patients more vulnerable to bacterial injection are a low white blood cell count, the presence of prosthetic heart valves or joints, and malnutrition, to name a few. Regardless of the vulnerability of the patient, if the bacterial injection causes clinical bacteremia, the death rate of even young and healthy children and adults is relatively high.

Patients are living longer with more prosthetic components, transplants, and vulnerabilities (as for example under the effects of chemotherapy) and therefore the risk posed by failure to swab the piston face will likely continue to increase over the next few decades unless technology such as that provided according to the present invention is broadly implemented. It is one of the purposes of the present invention to provide a simple, inexpensive luer valve disinfectant swab, which also can be employed to protect the luer valve face and the male luer itself from contamination between uses.

In addition it is not only the valve which can be readily contaminated. The male luer end of piggy back systems are also exposed after withdrawal from the luer valve and therefore these male luers should be covered between uses (which can often be 24 hours). U.S. Pat. No. 7,040,598 assigned to Cardinal Health discusses the problems with male luer contamination and discloses an elastomeric piston, which covers the male luer, as a solution. The face of the rebound able protective piston which protects the male luer would, like the face for the piston of the luer valve, have to be swabbed with disinfectant prior to each use, which, as has been discussed, is unreliable. Therefore with the disclosed system (as for example shown in FIG. 18b) of U.S. Pat. No. 7,040,598 would have the same limitations relating to “failure to swab” but doubled because now both the luer valve and the male luer cover would need to be swabbed. Replacement of the passive protection from contamination of the male luer provided by the male luer cap with active disinfection provided by disinfectant swabbing of the face of a piston over the male luer is asking the nurse to in essence “always remember to unload both guns” (disinfect two surfaces) prior to each access. The simple act of “failure to remember” now leaves two potentially lethal sources of bacteria for direct injection into the patient's blood vessel.

The present traditional approach of capriciously allowing free exposure of the piston face to contamination with deadly pathogens, with reliance on the diligence of the busy healthcare worker to remove the deadly organisms before use, is fundamentally flawed and cannot stand the test of time. Nurses are busier; patients are becoming more vulnerable as a function of transplantation, prosthetics, and/or chemotherapy, and organism more resistant. Each year the need to reduce the dependence of patient safety on the actions and diligence of the healthcare workers increases.

It is important to understand that there are several factors which may mitigate diligence with respect to swabbing of the luer valve immediately antecedent access. First, the skin interface has long been considered the primary source of catheter contamination so that many nurses do not believe that bacteria are likely to enter through the luer valve, many lack understanding relevant the complexity of bacterial contamination and incubation. The present inventor, upon noting a nurse in the ICU fail to swab a luer piston valve prior to connection reminded the nurse that the site must be swabbed first; the response of the nurse was to simply say that this site was a valve and that “valves did not need to be swabbed”. Even if the nurse knows the valve should be swabbed many do not think it to be of major importance, they may rationalize that after all, a few bacteria enter the bloodstream when one brushes the teeth. What they do not realize is that the bacteria on the faces of luer valve in hospitals are often potentially very deadly pathogens like Methcillin or Vancomycin resistant Staphylococci.

When substantial morbid and mortal risk in association with a high number of routine procedures is defined as a primary function of the diligence of a heterogeneous population of employees, a substantial degree of unnecessary injury to patients will inevitably result. For this reason, hospital patient safety is no longer considered a matter reasonably subject to procedural personal preference and personal diligence. Rather, patient safety should be controlled hospital wide with reliable passive technology. Present systems are designed such that the risks to patients and to hospitals are a substantial function of diligence of the employee performing the accesses. Since this diligence is largely uncontrollable, the risks associated with open piston luer valves are largely uncontrollable. The present inventor contends that it is unacceptable for hospitals to perform hundreds of thousands of accesses to patient's vascular system without controlling all of the reasonably controllable risks associated with the access procedure.

Although the open piston valves, because of their common and fundamental design flaws (from a microbiological perspective) such as the exposed circumferential space around the piston (see FIG. 1) and repetitively exposed piston sidewalls, will not survive the test of time, they are likely to remain in use in many parts of the world for some time. The more advanced split septum luer access technology as shown in U.S. Pat. No. 6,171,287, the contents of which is incorporated by reference as if completely disclosed herein, provides mechanical ant-infective capabilities and elimination of the open circumferential space and solves many of the above problems. However the safety of even this more advanced luer access technology can be improved by eliminations of routine exposure of the luer access interface and male luer to bacteria between uses.

The present invention serves to overcome the aforementioned problems of the prior art by providing a single inexpensive medical universal disinfectant swab, luer valve cover, and male luer cover called for example the LUER PROTECTION POUCH™, SWAB POUCH™ or SWAB POCKET™. In one embodiment the device serves as a luer valve swab and cover and male luer swab and cover. In another embodiment the device comprises a universal disinfectant skin and medical vial prep swab, a luer valve cover, a male luer cover, and a luer lock cover.

The universal device is configured to provide a wider range of multiple different functions thereby allowing the nurse to perform all of the basic functions of a swab including swabbing skin as well as all of the above steps of swabbing and covering male luers and a plurality of luer valve types with one unified device which protects the luer valve and the male luer between uses so that contamination by general exposure is prevented. Both devices are intended to be routinely carried by nurses in their lab coats and the more broadly functional universal device is intended as the only routine swab and valve cover which nurses need to carry. The device with extended functionality is capable of functioning as an essentially universally applicable disinfectant swab for skin, vials or access systems, a luer valve swab and cover (including for example a catheter terminal swab and cover, a Y site swab and cover, an extension set terminal swab and cover to name a few), and a male luer cover.

In one embodiment the invention comprises an expandable disinfectant swab member. In a preferred embodiment the disinfectant swab member is shaped to form a collapsed or flattened pocket, pouch, or tube and is sized to, when in an un-flattened state, snuggly and/or elastically fit over a luer valve and a male luer connector.

One embodiment comprises a disinfectant swab for protecting a luer valve wherein the swab comprises a pouch having an inner and outer surface the pouch further having shape memory and a disinfectant along at least one of the inner surface and the outer surfaces. In one example the shape memory can be provided by at least one portion of the pouch comprised of elastic material. The pouch can have at least one disinfectant along the outer surfaces so that the outer surface of the swab can be used to prep skin. The pouch can be sized to elastically grasp on at least one type of luer valve and in one embodiment to elastically grasp on a plurality of types of luer valves and/or to elastically grasp on a tube having the approximate outer diameter of a luer lock connector or another portion of a luer lock connector. The pouch can have a flattened configuration when stored and/or can be stored in a flat tear able sealed package. The pouch can be comprised of elastic foam. The pouch can define an end for receiving the valve and a passageway within the pouch, the passage way can be enlargeable along at least one dimension by compression of at least one portion of the pouch, as for example, by compression of opposing ends of the pouch. The pouch can have a first proximal blind sealed end and/or permanently closed proximal end and a closed but open able distal end. The open able distal end can be opened (as elastically opened) by compression of at least one portion of the valve. The distal end can elastically rebound to grasp the luer valve when the compression is released. The pouch can have an open able end (or another more proximal portion) of sufficient length, such that the pouch can still be dilated by compression after it has been installed on the valve. In an example a proximal portion of the pouch can have opposing edges which extend outwardly from the luer valve so that the passage way can be dilated to release the elastic grasp of the wall of the pouch upon the luer valve by the application of compression (as induced by the index finger and thumb) on the edges. The pouch can then be retracted without any (or any significant) retracting longitudinal force being transmitted to the valve or any adjacent catheter attached to the valve. The pouch can define oppositely facing walls, the walls can define a more central wall portion and more lateral wall portion, the more lateral portion having thicker walls than the more central portion so that dead space between the walls after the pouch has been installed over the valve is reduced

In one embodiment, the disinfectant swab member has two closed ends, a blind proximal end and an open able distal end for receiving the luer valve or male luer connector. The disinfectant swab member can comprise and/or contain and/or be coated with, and/or impregnated with, a disinfectant and/or one or more anti-infective agents such as chlorhexidine, alcohol, povidone iodine, or an antibiotic, to name a few. In one embodiment the disinfectant swab member can be comprised of medical grade foam which can be elastic. The entire disinfectant swab member can be comprised of elastic foam or the foam can be provided on the inside of the pouch. The disinfectant swab member can be comprised of medical grade foam and a coating or jacket of polymer and/or fabric may be provided on the outside or inside of the pocket. The memory and/or elasticity and/or the compressibility of the swab pocket over the valve (or the male luer) can retain the swab pocket over the luer valve and the male luer. This can, for example, be polymeric or fabric related memory for the flattened state in which the device may be packaged. In one embodiment the swab pouch has been coated internally with a mixture of chlorhexidine and 70% alcohol and externally with 70% alcohol alone (or the reverse with 70% alcohol alone on the inside and mixture of chlorhexidine and 70% alcohol on the outside). In one embodiment the pouch is entirely comprised of fabric which can be elastic and/or absorbent.

As noted, the universal swab-cover may be comprised of medical grade foam in the form of a foam pouch. The foam can be shaped to elastically retain the universal swab-cover over the luer valve. For example the universal swab-cover may be comprised of a single rectangle of thick elastic foam pouch comprised of opposing and/or contacting medical foam walls of about 1.5-2 cm thickness. The distal end of the foam pouch can be closed by expansion of the elastic foam at rest.

In an embodiment, the universal swab-cover can have a thinner foam wall proximally, the uncompressed and compressed foam at the distal (luer receiving end) can elastically hold the universal swab-cover in place. In addition, the distal end of the universal swab-cover provides a more rounded target configuration for direct receipt of the luer valve or male luer without the need (or with less need) for antecedent “change purse” type opening by compression of the ends, while at the same time protecting the interior from contact contamination. The elastic foam is compressed as the universal swab cover is placed over the valve and this provides one alternative type of elastic retention means to retain the universal swab cover over the valve.

In manufacture, a universal swab-cover comprised of medical grade foam can be formed by forming or cutting 3 cm by 4 cm rectangles of medical foam with a thickness of about 1.5-2.5 cm thick medical foam and then slitting the rectangles longitudinally to form a pouch with a blind proximal end. The pouches may alternatively be formed by longitudinally folding the sheet and the sealing (as by welding) along the two sides. Alternatively the universal swab-cover may be molded or woven to form the pouch.

In a preferred embodiment, the universal swab-cover is sized to be snugly and elastically retained over conventional female luer lock housing. Since many luer valves include a female luer lock housing distal the valve and since female luer lock housings are generally present surrounding the male luer with piggyback systems, the universal swab-cover can be sized to elastically hold onto the diameter of the female lure lock housing. According to one aspect of the present invention, the female luer lock housing provides a central universal target diameter for sizing an elastic universal swab-cover to assure retention.

As shown in U.S. Pat. No. 7,184,825 (which is incorporated by reference as if completely disclosed herein to provide background for the present invention), the diameter of the outer wall of the valve adjacent the distal end often approximates the diameter of a female threaded luer lock connector and according to the present invention this diameter can comprise one target diameter for the universal swab-cover in the expanded (open mouth) position. The universal swab-cover can be comprised of elastic material which is flattened at the open end and opened (like an elastic change purse) by compression of the finger and thumb perpendicular to the long axis of the closure. This configuration and mechanism of opening provides a high degree of flexibility for elastic retention on luer valves of various diameters since the lateral portions of the pouch (the length of which can be defined by the length of the closed end) can extend beyond the diameter of the valve and still provide retention in an elastic “fish mouth” configuration on the sidewalls of the valve. In one embodiment the material is thicker (or more rebound able) adjacent the sides than the center so that the sides which extend beyond a smaller diameter luer valve housing have a stronger rebound force for closure.

Alternatively the universal swab-cover can be packaged in a more “open pocket shape” with a distal opening being slightly closed or slightly open (with or without the interior and/or distal end filled with compressible foam). The universal swab-cover can comprise a narrower or more elastic neck with or without an enlarging distal end to provide a shape memory to providing tight engagement with the valve while allowing easy insertion over the valve. The neck or opening can be squeezed at the time of application over the valve to open it or enlarge the opening. The tight elastomeric neck with an enlarged distal end allows for a generally universal secure attachment to different shaped valves. The tight neck may also be employed to reduce the potential loss of a volatile disinfectant (if employed).

In an embodiment, a potion for attachment to the branch of a Y site such as a slit and/or Y site receiving channel and/or Y site receiving latch may be provided adjacent the open able end. The portion for attachment to the branch of a Y site may have at least one elastic portion for receiving the branch and for elastically holding the universal swab-cover over the branch to secure the valve to and over the valve or septum terminal adjacent the branch.

In an embodiment, the universal swab-cover is comprised entirely of non elastic material. In an example the inner layer can comprise a thin layer of absorbent cotton impregnated with a chlorhexidine and/or alcohol. An outer layer can be provided comprised of polyethylene terephthalate. The universal swab-cover may be specifically formed to fit over a specific valve shape. In the alternative, or in combination, a tether, latch or other connecting member may be provided for securing the universal swab-cover to the valve.

One method for protecting a patient from the transmission of bacteria through a medical valve, the valve defining a face and a valve stem comprises at least the step of placing an elastic pouch over the face and valve stem for a sustained period when the valve is not in use this can include the step of dilating the pouch as by squeezing the pouch and/or rubbing the pouch against the face. This can be accomplished for example by pressing against the outer surface of the pouch to press disinfectant from the pouch into forceful contact with the face.

One embodiment comprises a medical device for protecting from contamination by bacteria the distal end of a cannula comprising, an elastic pouch sized to be snuggly received and retained over the distal end of the cannula. The pouch can be a disinfectant swab a portion of which can be elastic. The pouch can be sized and configured to be elastically retained over the distal end of the cannula and, in one embodiment, first elastically opened and then received and retained over the distal end of the cannula. The pouch can contain and/or be coated with a disinfectant such as alcohol and/or chlorhexidine. In one embodiment the pouch has a different disinfectant along the inner surface than the disinfectant along the outer surface. For example the pouch can have alcohol on the inner surface and chlorhexidine on the outer surface so that chlorhexidine does not come into contact with the internal lumen of the male luer. The pouch can be configured so that no disinfectant comes into contact with the internal lumen of the male luer or can be configured so that the only disinfectant which can come into contact is a highly compatible disinfectant such as alcohol. The entire or the majority of the pouch can be comprised of elastic material or the pouch can be coated with elastic material (which may for example be coated by an application process such as immersion or spraying).

One embodiment comprises a medical device for protecting the proximal end of a cannula receiver and the distal end of a male cannula from contamination by bacteria, wherein the device comprises a flexible cover sized to be snuggly received and retained over the cannula receiver when the cannula receiver is not in use, and further sized to be snuggly received and retained over the distal end of the male cannula when the male cannula receiver is not in use. The cover can have the characteristic discussed supra for the pouch which protects the luer valve. The multifunctional swab cover can be further sized an configured to be snuggly received and retained over the distal end of the male luer when the luer valve is not in use and to elastically grasp onto the luer onto a luer lock connector over the distal end of the male luer.

One embodiment comprises a method for protecting a patient from the transmission of bacteria through a medical valve, the valve defining a face, and or protecting a patient from the transmission of bacteria through a male luer, the male luer having a distal end, the method can comprise the step of placing a pouch over the face of the valve for a sustained period when the valve is not in use and, placing a second pouch over the distal end of the male luer when the male luer is not in use. The method can further comprise the step of dilating at least one of the first and second pouches as by squeezing at least one of the first and second pouches.

In one embodiment the method for protecting a patient from the transmission of bacteria through a medical valve, and for protecting a patient from the transmission of bacteria through a male luer, comprises the step of:
a) placing a pouch over the face of the valve for a sustained period when the valve is not in use and,
b) swabbing the face of the valve, with the pouch
c) engaging the face of the valve with at least one of a male luer of a piggy back system and a male luer of a syringe,
d) flowing fluid through the valve,
e) disengaging the male luer from the face of the valve,
f) placing a first pouch over the distal end of the male luer and a second pouch over the face of the luer valve.

It is the purpose of the present invention to provide a system and method, which is designed to provide an inexpensive combined disinfectant swab, valve cover, and male luer cover which can provide this enhanced passive and active protection for a cost which does not greatly exceed the cost of the conventional prepackaged chlorhexidine and/or alcohol disinfectant swab itself.

It is the purpose of the present invention to provide a pouch can cover the either the face of the luer vale or the distal end of the male luer at the discretion of the user and without the transmission of torsion or longitudinal force which might loosen the to the luer valve or be transmitted to the vein.

It is the purpose of the present invention to provide a system and method, which is designed to provide an inexpensive combined disinfectant swab, valve cover, and male luer cover which can replace the conventional disinfection swabs in hospitals with more versatile and more universally applicable combined passive and active microbiological protection technology.

It is the purpose of the present invention to provide a flexible, collapsible combined disinfectant swab, valve cover, and male luer cover which has a flatted configuration when packaged so that nurses can easily carry and store large numbers of these as they do presently for conventional swabs.

It is the purpose of the present invention to provide a combined disinfectant swab, luer valve cover, and male luer cover which defines a blind pouch which has an open able distal end, which has memory for the closed state and spontaneously closes in the resting position.

It is the purpose of the present invention to provide a combined disinfectant swab, luer valve cover, and male luer cover which does not require threading onto the valve and which does not transmit torsion and/or longitudinal force to the valve or connecting catheter when applied over the valve.

It is the purpose of the present invention to provide a combined disinfectant swab, luer valve cover, and male luer cover which has an open able portion, which spontaneously holds onto the valve after the swab, has been placed over the valve.

It is the purpose of the present invention to provide a combined disinfectant swab, luer valve cover, and male luer cover which is comprised of an elastic pouch (which can be comprised of medical grade elastic foam) containing disinfectant, which disinfectant can comprise an anti-infective gel which fills the pouch.

It is the purpose of the present invention to provide a universal combined disinfectant swab, luer valve cover, male luer, and luer lock connector cover which is comprised of elastic foam which elastically retains the universal swab-cover over, male luer, luer lock connector and valves of various shapes.

It is the purpose of the present invention to provide a combined disinfectant swab, luer valve cover, and male luer cover which comprises a pouch and includes disinfectant on both the inner surface and outer surface of the pouch to provide a mechanism for the, “SWAB AND PROTECT™” or the, SWAB AND COVER™ procedure with a single swab system.

### BRIEF DESCRIPTION OF THE DRAWINGS

These, as well as other objects and advantages of this invention, will be more completely understood and appreciated by careful study of the following more detailed description of the presently preferred exemplary embodiments of the invention taken in conjunction with the accompanying drawings, in which:

FIG. 1A is an open piston valve of the prior art showing the piston face and the circumferential space around the piston.

FIG. 1B is a perspective view of a universal combined disinfectant swab, valve cover, and male luer cover called a Luer Protection Pouch™

FIG. 2A is a longitudinal section view through a luer receiving valve covered by a universal swab-cover comprised of medical grade foam.

FIG. 2B is a longitudinal cut section view of a luer lock connector with the distal end of the male luer (which can be connected to the distal end of a syringe or piggy back system) covered by the universal swab cover according to the present invention.

FIG. 3 is a transverse section view of the universal swab-cover through lines 3-3 of FIG. 2A.

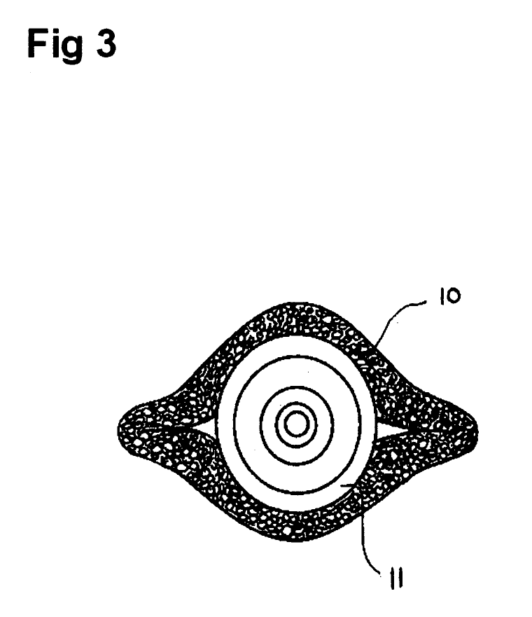

FIG. 4 is an action sequence showing one method of applying a universal swab-cover to a luer valve (the same procedure to apply over a male luer of the type shown in FIG. 2B).

### DESCRIPTION OF THE PRESENTLY PREFERRED EXEMPLARY EMBODIMENTS

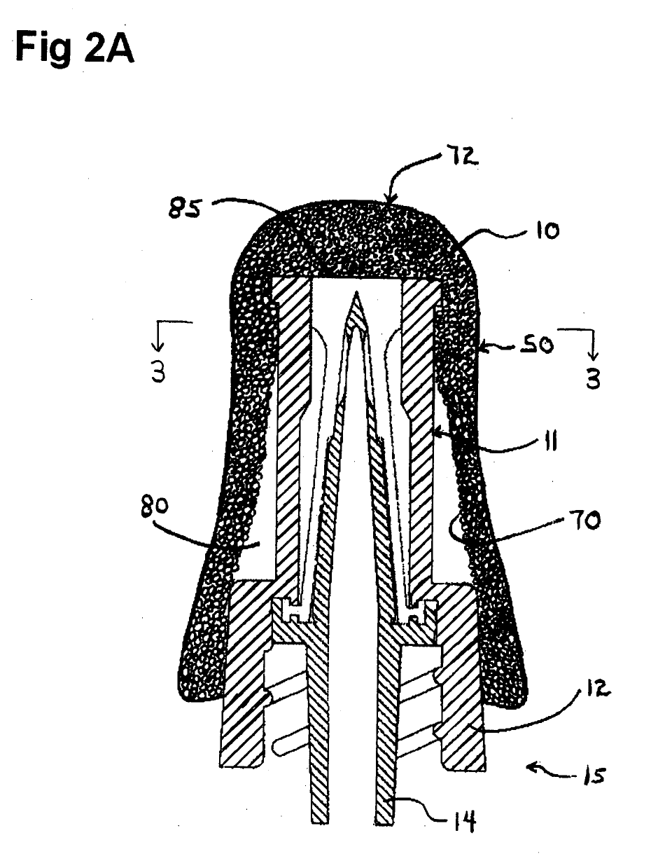

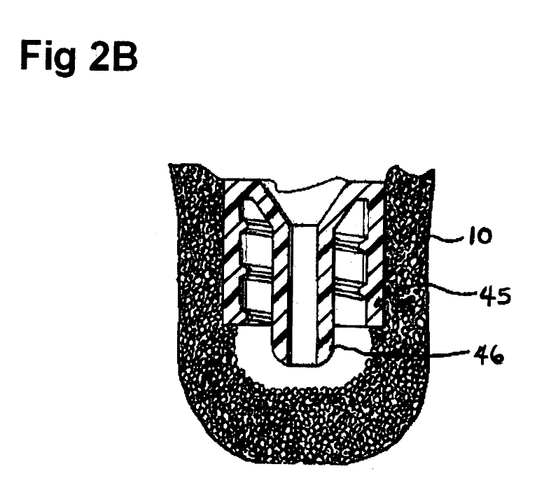

FIG. 2A, shows an embodiment of a combined disinfectant swab, valve swab and cover, and male luer swab and cover called a Luer Protecting Pouch™ or Universal Swab-Cover™ 10.

In a preferred embodiment the universal swab-cover 10 is shaped to form a collapsed or flattened pocket, pouch, or tube and is sized to, when in an un-flattened state, snuggly and/or elastically fit over a luer valve 11 (FIG. 2B) and particularly the female luer lock housing 12 which surrounds the male luer 14. The universal swab-cover 10 can include or be comprised of at least one component with shape memory. According to one aspect of the present invention, the female luer lock housing 12 of the male luer lock connector 15 provides a relatively universal target diameter for sizing the universal swab-cover 10 to assure retention over a plurality of luer valves.

One embodiment of the universal swab-cover 10 has two closed ends, a blind proximal end 40 and an open able distal end 42 for receiving the luer valve 11 (FIG. 2A) which in this figure is connected distally to a luer lock connector 15. The universal swab-cover 10 can comprise and/or contain and/or be coated with, and/or impregnated with, a disinfectant and/or one or more anti-infective agents such as chlorhexidine, alcohol, povidone iodine, or an antibiotic, to name a few. In an embodiment the swab pockets are brightly colored and/or the swab or package includes a bright trademark such as BD™ or 3M™. Since such swabs and covers will be ubiquitous in hospitals, this provides high visibility of the trademark throughout the hospital.

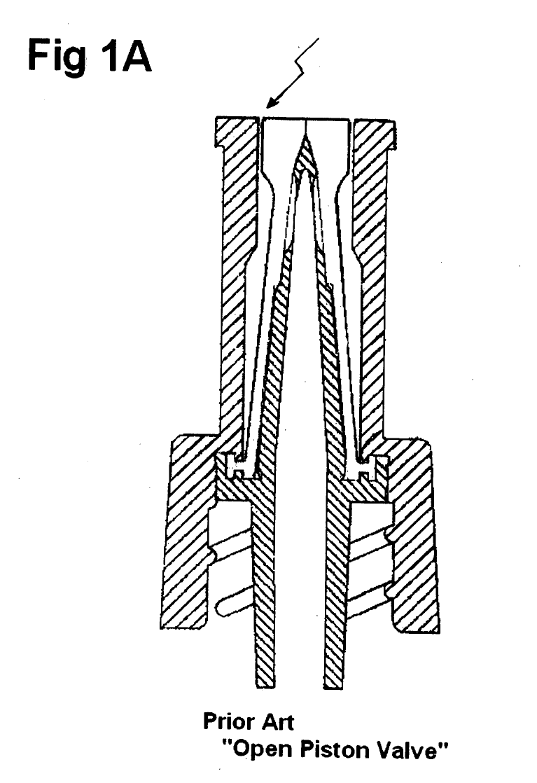

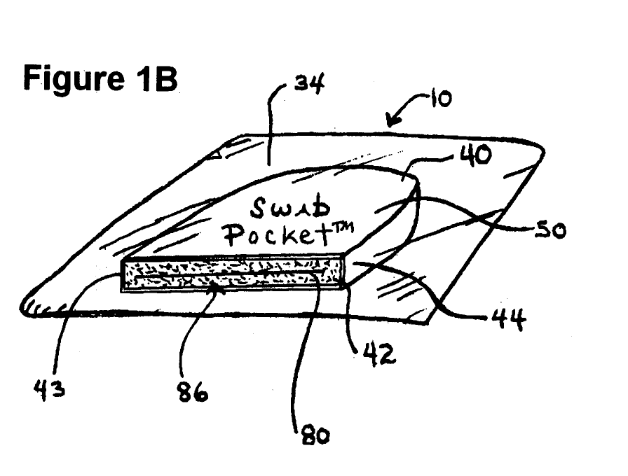

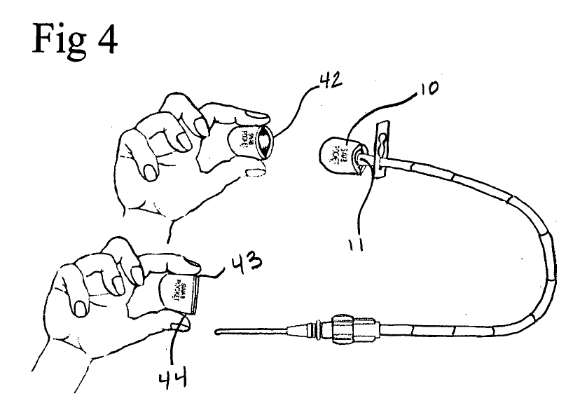

In one embodiment the universal swab-cover 10 can be clear, opaque, florescent, and/or translucent, if the swab-cover has a outer package, it can be packaged in a tear able clear, opaque, florescent, and/or translucent package 34 such as that shown in FIG. 1B (similar for example to the size of the tear able packages used for conventional alcohol prep swabs). The universal swab-cover 10 is preferably packaged with the open-able end 42 in a closed configuration as shown in FIG. 1B and the tear or serrated point of the package 34 adjacent the blind end 40 of the swab-cover 10. As shown in action sequence FIG. 4, during operation the sides 43 and 44 of the universal swab-cover 10 are squeezed, as by the index and thumb of the nurse, to open the open able end 42 of the universal swab-cover 10 for subsequent non frictional or low frictional application and self retentive covering of a luer valve 11. Before removal the universal swab-cover 10 can be used to swab the protected luer valve 11.

Disinfectant can also provided on the outside surface 50 of the universal swab-cover 10 the universal swab-cover 10 can also be used prepping skin or prepping drug vials and the like. A different disinfectant may be preferred for prepping skin than is preferred for prepping or protecting the luer valve or male luer so that a different disinfectant, mixture of disinfectants, or concentration of disinfectant(s) may be provided on the inside than that provided on the outside.

In one embodiment, the universal swab-cover 10 includes an absorbent inner layer 70 preferably comprised of medical grade foam or elastic fabric with or without another absorbent material such as cotton, with an outer less absorbent or non absorbent (water resistant) outer layer 72, which can for example be comprised of a polymer jacket such as for example polyethylene terephthalate, polyvinyl chloride, or polyolefin to name a few. The outer layer 72 may be comprised of a very smooth elastomere which can be compressible to provide for a component of mechanical anti-infectivity and to minimize pockets of incomplete contact during wiping of the valve face as the smooth elastomere engages the smooth silicone of the valve face. The outer layer 72 can, for example, be an optically clear elastic silicone sleeve, coating or molded component. The outer layer 72 can for example be molded with the fabric or molded into the fabric. Alternatively the entire universal swab-cover 10 may be comprised of an optically clear elastic silicone and/or of material with elastic shape memory such as the moldable elastomere sold under the trade name Zello™ marketed by Zeller International with an internal passage way 80 containing releasable disinfectant.

In an embodiment the universal swab-cover 10 is comprised of medical grade foam with an outer coating or jacket 72 compromised of a polymer and/or fabric. In one embodiment the surface is coated with 70% alcohol and/or another disinfectant with or without an alcohol component. Alternatively, the entire universal swab-cover 10 may be comprised of medical grade foam such as for example polyolefin foam or polyvinylchloride foam, and/or other suitable medical grade material which is at least partially impermeable to reduce evaporation of disinfectant liquid (if an evaporable liquid is used) from the universal swab-cover 10. The foam can be of open or closed cell type. In the open cell type, at least a portion of the cells may contain disinfectant and the cells may be incomplete or have internal wall perforations to allow the disinfectant to escape under compression. These perforations can be very small so that escape of the disinfectant is slow and controlled or the perforations can be large so that escape is rapid upon compression of the pocket against the valve face 85, for example.

Presently most luer valves have at least one portion which has a diameter of about 1 cm. and this can provide one of the target diameters of the opened universal swab-cover 10 adjacent the distal end or mouth 86 (FIG. 1B) of the universal swab-cover 10. In an embodiment, the unstretched wall thickness is about 1-5 mm and the width along the flattened axis of the distal end is about 1.5-3.0 cm so that the expanded internal diameter, when the universal swab-cover 10 is opened by compression applied by the index finger and the thumb along the distal end 86 renders a preferred target diameter range of about 7 mm-15 mm. Other sizes of swab-covers can be provided.

An example of an alternative embodiment for protecting the joint of connected IV tubing comprises an elastic swab-cover configured as a cylinder which can be flattened with a slit which extends along the longitudinal axis on one side of the cylinder to the mid point of the cylinder. This cover can be squeezed at opposite ends of the cylinder to open the slit for installing over a IV tubing to tubing or male luer to luer valve connection or other connection to protect the connection site (and the connection ends) from contamination at the joint which contamination can otherwise enter the system form the edges of the system form the edges of the tubing if the connection is disconnected and reconnected. This is particularly for use in patients such as those with very low WBC counts who need maximal protection or for IV tubing (e.g. hub to hub) connection sites which are highly vulnerable to colonization such as those associated with the administration of total parenteral nutrition or lipid carried drugs such as Propofol. This provides an elastic, antiseptic, self retaining protector over vulnerable hub to hub connection sites when the hubs are connected.

Although the presently preferred embodiments have been described, it will be obvious to those skilled in the art that various changes and modifications can be made without departing from the invention. While the invention has been described in connection with what is presently considered to be the most practical and preferred embodiments, it is to be understood that the invention is not to be limited to the disclosed embodiments.

1. A medical device comprising for protecting from contamination by bacteria the distal end of a cannula comprising, an elastic pouch sized to be snuggly received and retained over the distal end of the cannula.

2. The medical device of claim 1 wherein the pouch is a disinfectant swab.

3. The medical device of claim 1 wherein a portion of the pouch is water resistant.

4. The medical device of claim 1 wherein the pouch is sized and configured to be elastically retained over the distal end of the cannula.

5. The medical device of claim 1 wherein the pouch sized and configured to be elastically opened and then received and retained over the distal end of the cannula.

6. The medical device of claim 1 wherein the pouch contains an antiseptic such as at least one of alcohol and chlorhexidine.

7. The medical device of claim 1 wherein the pouch is coated an antiseptic such as at least one of alcohol and chlorhexidine.

8. The medical device of claim 1 wherein the pouch has a first proximal blind end and a closed but open able distal end.

9. The medical device of claim 1 wherein the pouch has a proximal end and an open able distal end and wherein the pouch is configured such that the open able distal end can be opened by compression of at least one portion of the pouch.

10. The medical device of claim 1 wherein the pouch has an inner and outer surface, an water resistant component along the outer surface.

11. The medical device of claim 1 the pouch has an inner and outer surface and disinfectant along at least one of the inner surface and the outer surfaces and a different disinfectant along the inner surface than the disinfectant along the outer surface.

12. The medical device of claim 1 the pouch has an inner and outer surface and a disinfectant along the inner surface comprised of alcohol.

13. The medical device of claim 1 wherein the pouch has a flattened configuration when stored.

14. The medical device of claim 1 wherein the pouch is stored in a flat tear able sealed package.

15. The medical device of claim 1 wherein the pouch is comprised of elastic foam.

16. The medical device of claim 1 wherein the cannula is a male luer.

17. The medical device of claim 1 wherein the cannula is a male luer having a distal end, the pouch being sized and configured to be elastically opened and then received and elastically retained over the distal end of the male luer.

18. The medical device of claim 17 wherein the pouch is further sized and configured to elastically grasp on at least one type of luer valve, the luer valve having a proximal outer face, the pouch covering the outer face when the pouch elastically grasps the valve.

19. The medical device of claim 17 wherein the pouch is further sized and configured to grasp on a plurality of types of luer valves.

20. The medical device of claim 17 wherein the pouch is further sized and configured to elastically grasp on a tube having the approximate outer diameter of a luer lock connector.

21. The medical device of claim 17 wherein the pouch is further sized and configured to elastically grasp on a luer lock connector.

22. The medical device of claim 1 wherein the pouch is a disinfectant swab sized to be snuggly received and retained over the distal end of the male luer when the male luer is not in use.

23. The medical device of claim 1 wherein the cannula is a male luer surrounded by a luer lock connector, the luer lock connector having a distal end.

24. The medical device of claim 23 wherein the pouch is configured to elastically rebound to grasp the luer lock connector when the compression of the pouch is released.

25. The medical device of claim 23 wherein the pouch defines a closed distal end, the distal end of the pouch being elastic and open able by compression, the pouch rebounding to elastically grasp adjacent the luer lock connector when the compression of the pouch is released.

26. The medical device of claim 23 wherein the pouch has a portion of sufficient length, such that the pouch can be dilated by compression after it has been installed adjacent the luer lock connector.

27. The medical device of claim 23 wherein the pouch has opposing edges which project outwardly after the pouch has been installed over the luer lock connector so that the passage way can be dilated to release the elastic grasp of the wall of the pouch upon the luer lock connector by the application of compression induced by an index finger and thumb on the projecting edges of the pouch.

28. A medical device for protecting the proximal end of a cannula receiver and the distal end of a male cannula from contamination by bacteria, the device comprising: a flexible cover sized to be snuggly received and retained over the cannula receiver when the cannula receiver is not in use, the cover being further sized to be snuggly received and retained over the distal end of the male cannula when the male cannula receiver is not in use.

29. The medical device of claim 28 wherein the cover is a disinfectant swab.

30. The medical device of claim 28 wherein the cover is configured, to exhibit elastic behavior.

31. The medical device of claim 28 wherein at least a portion of the cover is elastic.

32. The medical device of claim 28 wherein the cover is a pouch.

33. The medical device of claim 28 wherein the cover is configured to be elastically retained over the proximal end cannula receiver and the distal end of the cannula.

34. The medical device of claim 28 wherein the cover is comprised of a pouch, the pouch further has a blind proximal end and an open able but closed distal end.

35. The medical device of claim 28 wherein the cover has an inner and outer surface and at least one disinfectant along at least one of the inner surface and the outer surfaces.

36. The medical device of claim 28 wherein the cannula is a male luer and the cannula receiver is a luer receiving valve having a proximal face.

37. The medical device of claim 36 wherein the cover is a pouch sized to be snuggly received and retained over at least the proximal face of the luer valve for protecting the proximal face of the luer valve when the luer valve is not in use, the pouch being further sized to be snuggly received and retained over the distal end of the male luer when the male luer is not in use.

38. The medical device of claim 37 wherein the pouch is configured, to exhibit elastic behavior.

39. The medical device of claim 37 wherein the at least a portion of the pouch is comprised of elastic material.

40. The medical device of claim 37 wherein the pouch is sized to elastically grasp onto the luer valve over the proximal end of the valve and to elastically grasp onto a luer lock connector over the distal end of the male luer.

41. The medical device of claim 37 wherein the pouch has a flattened configuration when stored.

42. A method for protecting a patient from the transmission of bacteria through a medical valve and for protecting a male luer from contamination by bacteria, the valve having a proximal face and the male luer having a distal end, the method comprising the step of placing a first pouch over the proximal face of the valve for a sustained period when the valve is not in use and, placing a second pouch, which is identical to the first pouch, over the distal end of the male luer when the male luer is not in use.

43. The method according to claim 42 further comprising the step of dilating at least one of the first and second pouches.

44. The method according to claim 42 further comprising the step of dilating at least one of the first and second pouches by squeezing the pouch.

45. A method for protecting a patient from the transmission of bacteria through a medical valve and for protecting a male luer from contamination by bacteria, the valve having a proximal face and the male luer having a distal end, the method comprising the steps of:
a) placing a first disinfectant pouch over the face of the valve for a sustained period when the valve is not in use and,
b) swabbing the face of the valve, with the pouch over the face of the valve
c) removing the first disinfectant pouch,
d) engaging the face of the valve with at least one of, a male luer of a piggy back system and a male luer of a syringe,
e) flowing fluid through the valve,
f) disengaging the male luer from the face of the valve,
g) placing a second disinfectant pouch, over the distal end of the male luer and a third disinfectant pouch over the face of the luer valve.

46. A method for protecting a male luer from contamination by bacteria, the male luer having a distal end, the method comprising the steps of:
a) engaging the face of the valve with a male luer of a syringe,
b) injecting a first dose of medication through the valve,
c) disengaging the male luer from the face of the valve,
d) placing an elastic disinfectant pouch, over the distal end of the male luer.

47. A method for protecting a hub-to-hub connection of medical tubing from contamination by bacteria, the method comprising steps of;
a) connecting the tubing
b) placing an elastic cover containing disinfectant over the connection site after the connection has been made.
